(19) United States
(12) Patent Application Publication
(10) Pub. No.: US 2005/0014176 A1
(43) Pub. Date: Jan. 20, 2005

(54) Method for quantitative end-point PCR

(76) Inventors: Roy R. Swiger, Indialantic, FL (US); Eric J. Emmerling, Melbourbe, FL (US)

Correspondence Address: BLACKWELL SANDERS PEPER MARTIN LLP, 720 OLIVE STREET, SUITE 2400, ST. LOUIS, MO, 63101, US

(21) Appl. No.: 10/848,618

(22) Filed: Oct. 17, 2003

Related U.S. Application Data
(60) Provisional application No. 60/419,879, filed on Oct. 18, 2002.

(51) Int. Cl. C12Q 1/68; C12P 19/34

(52) U.S. Cl. 435/6; 435/91.2

(57) ABSTRACT

The present invention provides a sensitive and robust analytical approach to identifying and quantifying multiple pathogens within a single complex environmental sample. Numerous nucleic acid signatures may be screened and quantified within a single reaction tube using polymerase chain reaction (PCR) and chromatographically analyzing the amplification products with microchannel fluidic (e.g. Agilent 2100 Bioanalyzer, or Caliper AMS-90) or reverse-phase ion-pairing high-performance liquid chromatography RP IP HPLC (e.g. Transgenomic WAVE) instruments. The method may be employed in a multiplex fashion to allow identification and quantification of multiple combinations of up to five different nucleic acid signatures simultaneously within a single multiplex PCR reaction tube. This approach is quantitative across a dynamic range of up to five orders of magnitude. This method is suitable for target nucleic acid analysis in medium or high-throughput contexts such as routine clinical diagnotics or environmental monitoring. The method is also suitable for pathogen monitoring or surveillance in a biodefense context.

### CROSS-REFERENCE TO RELATED APPLICATIONS

This application claims benefit of U.S. Provisional Application No. 60/419,879, filed on Oct. 18, 2002.

### BACKGROUND OF INVENTION

The present invention is related generally to a novel PCR method and, more specifically, to a novel method for quantitative analysis of the initial amount of target nucleic acids in a sample.

Currently, there is a strong need to develop robust quantitative diagnostic technologies for use in multiple contexts (e.g., clinical diagnostics, forensics, or the United States Postal Service domestic bioweapon monitoring). Because DNA signatures are more stable than protein or RNA in most environments, and because DNA can be amplified specifically, a quantitative multiplex PCR method is well-suited to the screening of samples from any of the above-described sources, among others. Moreover, it is conceivable that some government agencies or commercial businesses will require analysis of tens of thousands of samples at any given time. A robust, inexpensive method will provide the high-throughput screening compatibility that is desired in such instances.

The application of multiplex PCR analysis within clinical diagnostic applications can reduce the cost of analysis, and is at least two orders of magnitude more sensitive than an equivalent immunological application (see Henchal et al., 2001). Moreover, the speed with which the PCR assay can be conducted is superior to clinical assays that have equivalent sensitivities, such as live-culture of the samples in question (see Henchal et al., 2001).

Polymerase chain reaction (PCR) is an in vitro synthesis method that uses a thermal-stable polymerase enzyme and can be used for the synthesis of specific target nucleic acid sequences. The PCR method uses oligonucleotide primers that hybridize to opposite strands of a nucleic acid molecule and flank a specific region of the target nucleic acid that is to be amplified. During a single cycle of amplification, the primers are annealed to the target, extended using a thermostable DNA polymerase enzyme, and, finally, denatured. This cycle is repeated a number of times until an adequate amount of the target nucleic acid is produced. The use of automated thermal cyclers allows for a repetitive series of amplification cycles, each involving target denaturation, primer annealing, and the extension of annealed primers by DNA polymerase, to proceed conveniently, resulting in the exponential accumulation of amplification products (i.e. DNA copies of the specific target region of DNA whose termini are defined by the 5′ end of the primers). A specific target region of DNA sequence may be selectively amplified by a factor of 10⁹ using PCR. The PCR method of nucleic acid amplification has been well described in the art (see e.g. Mullis and Faloona, Methods in Enymol. Vol. 155, pg. 335 (1987)). Further improvements in the basic PCR method have been disclosed in U.S. Pat. Nos. 4,683,202; 4,683,196; and 4,800,159, each of which are incorporated by reference herein in its entirety.

Multiplex PCR (MP PCR) refers to the simultaneous PCR amplification of multiple target nucleic acid sequences within a single closed reaction tube. MP PCR analysis is desirable when the availability of the sample is sparse, the sample needs to be conserved for other reasons, or when samples are present in great numbers (as is the case in high-throughput systems (HTS) exclusionary screening applications). MP PCR reaction conditions are difficult to develop and optimize because the sequence differences among the numerous targets and corresponding sets of primer pairs often require different optimal reaction conditions (e.g., melting-temperatures (Tₘ) or salt concentrations). To some extent these differences may be mitigated using commercial “primer designing” software programs, and, or by selectively designing each set of primer pairs to have similar melting temperatures. In addition, improved fidelity of MP PCR assays may be achieved by reducing primer concentrations in addition to using proprietary commercial chemistries (e.g., Gibco SuperMix). Primer concentrations, sequence-context differences among primer sets, and differences in Tₘ are, however, the most significant limiting factors in determining the total number of targets that can be screened within a single PCR reaction.

Real-time PCR refers to a homogenous PCR assay that permits continuous fluorescent monitoring of the kinetic progress of the amplification reaction. Methods of conducting real-time PCR are well known in the art and a number of systems are available commercially (see e.g. Higucho et al., “Kinetic PCR Analysis: Real-time Monitoring of DNA Amplification Reactions,” Bio/Technology 11:1026-1030 (1993)). In its most commonly used embodiment, real-time PCR employs a specially labeled oligonucleotide probe in addition to the primers. The probe is designed such that it binds to the target but is degraded during the amplification or polymerization reaction, yielding a fluorescent signal when the DNA polymerase makes a copy of the target. The data can be captured in real-time by detecting and or measuring the fluorescent signal. The requirement for specialized fluorescent probes and real-time detection systems can greatly increases the costs associated with using real-time PCR.

Despite its greater expense, real-time PCR has become the method of choice in bioanalytical labs because it appears to be the best method of providing quantitative measurements of the starting target nucleic acid amounts in an amplified sample. Specifically, real-time PCR quantitation is achieved by monitoring the reaction time course as it enters the log phase and then extrapolating the amplification cycle in which the fluorescent signal first crosses a detection threshold (C_{T}). The C_{T} is determined differently with each technology and supporting software. One definition used commonly by researchers defines the C_{T} as the level of fluoresencet signal that exceeds 10 standard deviations of the background fluorescence associated with each sample or all samples during the early rounds of amplification and before the samples enter the log-phase of the reaction. A standard series of known samples (usually serially diluted by a factor of ten-fold) are used to determine the linear relationship between cycle number C_{T} for a data sample set using a semi-log X-Y coordinate plane (regression analysis). By comparing an unknown sample C_{T} value to the linear regression plot from a series of known standards, it is possible to estimate the quantity of the starting material (mass or number of copies) of the target nucleic acid in each unknown sample. Yet, as mentioned above, because the method can involve real-time monitoring of the PCR time-course for a sample and set of standards, it requires acquisition of large kinetic data sets in order to extrapolate the quantitative result. Here, we describe another quantitative method that is amenable to high-throughput analysis and requires a single measurement of the results of the amplification products from a sample often referred to as “end-point” (i.e. after the log phase). where the resulting PCR amplification products are at the highest concentration.

Multiplex real-time PCR and associated instrumentation have been reported for use in identifying or screening multiple microbes from within complex environmental mixtures. (see e.g. Vet et al., “Multiplex detection of four pathogenic retroviruses using molecular beacons,” Proc. Natl. Acad. Sci. USA 96:6394-6399 (1999)). Multiplex real-time PCR, however, presents the same difficulties related to primer sequences associated with standard MP PCR. Further, multiplex detection in the real-time PCR method is limited by the problem of spectral overlap of the different fluorescent signals. The use of multiple fluorescent labels also results in an increased basal-level of fluorescence within the reaction, which results in lower signal to noise ratios. Because of these problems, the maximum number of targets that can be quantified using current real-time PCR methods and instrumentation is effectively limited to four.

The present invention provides a quantitative PCR method that overcomes the above-described limitations of real-time PCR. Most significantly, the disclosed invention does not require fluorescent labeling or detection. Nor does it require kinetic monitoring of the PCR reaction time course. Rather, the present invention provides a method wherein a PCR reaction is run until its end-point, or plateau phase, and the unlabeled target nucleic acid is then quantified using reverse-phase ion-pairing high performance liquid chromatography (RP IP HPLC) and/or micro-channel fluidic instrumentation (MCF). Because no fluorescent labeling or detection is required, cost is greatly decreased with respect to the typical real-time PCR system. Furthermore, because it does not rely on fluorescent detection, the present invention provides a method of quantitative multiplex PCR that is not limited to four targets. The present quantitative end-point multiplex PCR assay is compatible with standard medium throughput screening (MTS) or high-throughput screening (HTS) requirements. Thus, the present approach is highly advantageous when dealing with quantitative assay situations that require: a) sample conservation; b) screening for multiple targets within a single sample; and c) HTS of large numbers of complex samples.

### SUMMARY OF INVENTION

The present invention is directed to a method for quantifying the amount of target nucleic acid in a sample comprising determining the area under the curve of an elution peak provided by separation analysis of a PCR amplification product resulting from said target nucleic acid and extrapolating the starting amount of the target nucleic acid in said sample by comparing said area under the curve with a standard.

In one embodiment, the present method includes the following steps: a) providing a sample having one or more target nucleic acids and one or more pairs of nucleic acid primers selected to amplify the target nucleic acids; b) subjecting the sample to PCR amplification conditions; c) introducing the sample into a chromatographic separation device and detecting the elution peaks of each PCR amplification product in the sample; and d) comparing the area of each PCR amplification product elution peak to the area of a PCR amplification product elution peak representing the PCR amplification of a standard comprising a known amount of each target nucleic acid, thereby determining the amount of each target nucleic acid in the sample.

In another embodiment, step d) of the above-described method further includes: extrapolating the amount of target nucleic acid in the sample from the area of the PCR amplification product elution peak by extrapolation from a plot comprising a first axis corresponding to the known amount of target nucleic acid in each standard and a second axis corresponding to the area of the PCR amplification product elution peak of each standard.

In some embodiments of the present invention, the PCR amplification conditions of the method include carrying out the PCR reaction until it reaches an end-point beyond the log phase of amplification (e.g. in the plateau phase).

In other embodiments, the present invention provides a method of quantifying the amount of multiple target nucleic acids in a sample. This method includes the following steps: a) providing a sample having two to ten (preferably 4 to 6) target nucleic acids and two to ten pairs of nucleic acid primers selected to amplify the target nucleic acids; b) subjecting the sample to PCR amplification conditions; c) introducing the sample into a chromatographic separation device and detecting elution peaks of each PCR amplification product in the sample; and d) comparing the area of each PCR amplification product elution peak to the area of a PCR amplification product elution peak representing the PCR amplification of a standard comprising a known amount of each target nucleic acid, and thereby determining the amount of each of the two to ten target nucleic acids in the sample. In some embodiments, the above-described quantitative multiplex PCR method may be used to quantify greater than 10 different target nucleic acids in a sample.

Furthermore, in some embodiments, each of the two to ten (or more) different target nucleic acids in the sample is derived from a different organism.

In yet further embodiments, the above-described quantitative PCR methods are carried out with unlabeled target nucleic acid primers, or without any fluorescent labeled probes Alternatively, fluorescently labeled primers may be used to increase sensitivity, in which case the labels are separated before detection there is no spectral overlap.

In still other embodiments of the-quantitative PCR methods of the present invention, the amount of the target nucleic acids in the sample is between about 10 femtograms and about 100 femtograms. In other embodiments, the amount of the target nucleic acid in the sample is less that 10 femtograms. Furthermore, in some embodiments, the known amount of target nucleic acid in the three or more standards ranges from about 10 femtograms to about 10,000 femtograms. Standards with less than 10 femtograms or more than 10,000 femtograms may likewise be used with the method of the present invention.

Other embodiments of the method of the present invention are carried out with samples further including one or more non-target nucleic acids. In some embodiments, the sample may be even more complex, including non-target nucleic acids from organisms other than the source of the target nucleic acid.

In another embodiment of the present invention, the quantitative PCR method may be carried out using a reverse-phase ion-pairing HPLC column as the separation device. In yet another embodiment, the separation device may be a micro-channel fluidic device.

In another embodiment of the present invention, the quantitative PCR method is carried out such that the length of the PCR amplification product generated from the target nucleic acid is between about 50 nucleotides and about 500 nucleotides. In still another embodiment of the present invention, the length of the PCR amplification product generated from the target nucleic acid is between about 75 nucleotides and about 250 nucleotides.

In a multiplex embodiment of the present invention, the PCR amplification products generated from the multiple different target nucleic acids differ in size by at least 10 nucleotides.

### BRIEF DESCRIPTION OF DRAWINGS

FIG. 1: (A) RP IP HPLC elution profile for sample of target standard dilution series following PCR; (B) chart of numerical data corresponding to target standard PCR amplification product elution peaks at ˜8.15 minutes.

FIG. 2: (A) Quantitative plot of target nucleic acid standard dilution series for “target 4” versus PCR amplification product RP IP HPLC elution peak areas; (B) real-time PCR analysis of same standard dilution series.

FIG. 3: (A) Quantitative plot of target nucleic acid standard dilution series for “target 3” versus PCR amplification product RP IP HPLC elution peak areas; (B) real-time PCR analysis of same standard dilution series.

FIG. 4: (A) Quantitative plot of target nucleic acid concentrations for targets 1 and 2, versus elution peak area as determined by micro-channel fluidics; (B) Quantitative plot of target nucleic acid concentrations for targets 3 and 4, versus elution peak area as determined by micro-channel fluidics.

### DETAILED DESCRIPTION

The present invention is directed to an end-point PCR method that allows sensitive quantitative analysis of the initial amounts (i.e. copy numbers) of target nucleic acid in a sample without fluorescent probes or real-time reaction monitoring. The practice of this invention requires, unless otherwise indicated, the standard techniques of molecular biology, microbiology, and nucleic acid analysis, which are well known to those skilled in the art.

The quantitative end-point PCR method disclosed herein may be utilized in a wide range of applications where quantitative analysis of nucleic acids is required. Perhaps most significantly, this method may find use in any application involving detection and quantitative analysis of pathogens. Generally, the applications for which the present invention is suitable include quantitative PCR monitoring of pathogens, microbes, bacteria, viruses, fungi and mold, in the context of bioweapon defense, agricultural pathogen monitoring, environmental monitoring, or traditional clinical diagnostics. The present invention may be used in the quantitative detection of the three main categories of pathogenic threats identified by the CDC (see Table 1 below), among others.

The general method for quantitative end-point PCR includes the following steps: a) providing a sample having one or more target nucleic acids and one or more pair of nucleic acid primers selected to amplify the target nucleic acids; b) subjecting the sample to PCR amplification conditions; c) introducing the sample into a chromatographic separation device and detecting elution peaks of each PCR amplification product in the sample; and d) comparing the area of amplification of the sample to the area of amplification of a standard comprising a known amount of each target nucleic acid, thereby determining the amount of each target nucleic acid in the sample.

The present method is not limited to a singleplex embodiment. Quantitative multiplex PCR of different target nucleic acids in the same sample may also be carried out using this method. Because the method utilizes chromatographic separation devices to quantify the end-point amplification products, the present method does not have the problem of spectral overlap found in multiplex real-time PCR. In the multiplex embodiments, primers are selected to produce PCR amplification products of each of the different target nucleic acids that may be distinguished by chromatography. In some embodiments, the different PCR amplification products differ in length by an amount (e.g. at least 10 nucleotides) such that their respective elution peaks do not overlap. Consequently, using HPLC conditions well-known in the art, the elution peak areas of multiple different PCR amplification products from a single multiplex sample may be determined simultaneously. In some embodiments of the multiplex quantitative PCR method, multiplex standards containing known starting amounts of each of the different nucleic acids are used.

In the present invention, a sample or specimen is provided that is suspected or known to contain a particular target nucleic acid of interest. In some embodiments, the target nucleic acid is a single-stranded RNA that must first be reverse-transcribed into double-stranded DNA. The sample may be prepared using any of the standard methods well-known in the art for isolating and preparing nucleic acids for PCR amplification. Samples may be obtained from any organism or source from which DNA or RNA may be derived. For example, the sample source may be pathogenic spores obtained via standard sample swabbing techniques.

In some embodiments, the sample may be highly complex. i.e. containing large quantities of non-target nucleic acids in addition to the target nucleic acid to be quantified. Interference by non-target nucleic acids may be avoided using PCR primer sequence selection techniques well-known in the art.

One advantage of the present invention is that it may be carried out using standard PCR amplification conditions. Amplification conditions that must be determined for any given PCR reaction include temperature and times for annealing, reaction and denaturing steps, as well as the choice of thermostable enzymes and/or salt and buffering conditions. A wide variety of PCR amplification conditions are well-known to those of ordinary skill in the art. Different conditions may be used with the present method depending on the target nucleic acid to be quantified. In some embodiments, PCR amplification conditions may include reverse transcription of single-stranded RNA nucleic acids. PCR methods that include reverse transcription of RNA are well-known in the art.

The primers may be selected according to any of the PCR primer selection methods well-known in the art. Primer selection is dependent on a number of factors, including reaction temperature, sequences of the target annealing site, and overall complexity of the target (and non-target) sequence in the sample. In the multiplex PCR embodiment, different primers for the different targets should be selected for specific binding only to their intended targets under the particular multiplex PCR amplification conditions.

In some embodiments of the present invention, the primers are not labeled and the amplification products may be detected based on the standard method of detecting UV absorbance near 260 nm. In alternative embodiments, however, the primer may be labeled. In one alternative embodiment, fluorescent labeling of the primers may be used to allow PCR elution peak detection at lower concentrations. In some embodiments, fluorescent label may be incorporated to allow detection of the PCR amplification products using quantitative hybridization techniques well-known in the art. Alternatively, radiolabeling of PCR primers may be used with the present invention, especially in applications where extremely high sensitivity is desired.

TABLE 1
CDC List of Thread Pathogens

| Category A | Category B | Category C |
|---|---|---|
| Bacillus antracis | Burkholderia pseudomallei | Emerging Infectious |
| (anthrax) | | Diseases: Nipah |
| | | virus and additional |
| | | hantaviruses. |
| Clostridium botulinum | Coxiella burnetti (Q fever) | Tickborne |
| | | hemorrhagic |
| | | fever viruses |
| Yersinia pestis | Brucella species | Crimean-Congo |
| | (brucellosis) | hemorrhagic fever |
| | | virus |
| Variola major | Burkholeria mallei | Tickbornes |
| (smallpox) and | (glanders) | encephalitis viruses |
| other pox viruses | | |
| Francisella tularensis | Ricin roxin (from Ricinus | Yellow fever |
| (tularemia) | communis) | |
| Viral hemorrhagic | Epsilon toxin of | |
| fevers: | Clostridium perfringens | |
| Arenaviruses | Staphylococcus enterotoxin | |
| LCM, Junin virus, | B | |
| Machup virus, | Typhus fever (Rickettsia | |
| Guanarito virus | prowazekii) | |
| Lassa Fever | Food and Waterborne | |
| Bunyaviruses | Pathogens: | |
| Hantaviruses | Bacteria: | |
| Rift Valley Fever | Diarrheagenic E. coli | |
| Flaviruses | Pathogenic Vibrios | |
| Dengue | Shigella species | |
| Filoviruses | Salmonella | |
| Ebola | Listeria monocytogenes | |
| Marburg | Campylobacter jejuni | |
| | Yersinia enterocolitica | |
| | Viruses | |
| | Calciviruses | |
| | Hepatitis A | |
| | Protozoa | |
| | Cryptosporidium parvum | |
| | Cyclospora cayatensis | |
| | Giardia lamblia | |
| | Entamoeba histolytica | |
| | Toxoplasma | |
| | Microsporidia | |
| | Additional viral | |
| | encephalides: | |
| | West Nile Virus | |
| | LaCrosse | |
| | California encephalitis | |
| | VEE | |
| | EEE | |
| | WEE | |
| | Japanese Encephalitis Virus | |
| | Kysanur Forest Virus | |

The quantitative PCR method of the present invention may be performed with existing medium through-put (MTS) or high-throughput (HTS) analytical platforms well-known to those of skill in the art. For example, the present invention may be used with formatted reaction tubes, bar-coding, and decoder plates, permitting the cataloguing of analyzed samples for long-term storage. In addition to easy cataloguing of samples, MTS or HTS compatibility means that other elements of an analysis stream can be optimized, automated or streamlined with little human manipulation. Any chromatographic separation device amenable to quantitation of DNA may be used with the method of the present invention. Many such devices are well-known to those of skill in the art, including reverse-phase ion-pairing HPLC, and micro-channel fluidic devices. For example, the Transgenomic WAVE RP-IP HPLC system may be employed in the present method. Using this system, the method allows quantitative analysis across a dynamic range of at least 5 orders of magnitude. Examples of compatible micro-channel fluidic devices include the Agilent Bioanalyzer 2100, or the Caliper AMS-90.

Alternatively, any analytical platform that allows quantitative analysis of DNA may be used in the method of the present invention to analyze the amount of end-point PCR amplification products. For example, microarrays or microsphere flow-cytometry (e.g. Luminex LabMap System) have been used in assays to identify single-nucleotide polymorphisms in multiplex fashion. The above analytical platforms may be used in instances in which primer design contraints result in putative amplification products that cannot be separated or identified by size.

Generally, the present method achieves quantitative analysis of the initial target nucleic acids by calculating the integral of the area under the curves produced by the chromatographic analysis of PCR amplification products. As in real-time PCR, quantitation of unknowns requires the generation of a standard curve using previously quantified starting material. By plotting the area(s) under the curve against the known starting copy number, the value of unknowns may be determined.

In some embodiments, the PCR amplification of the standards is conducted in separate tubes contemporaneously with the sample. Alternatively, depending on the stability of the system, one may amplify and chromatographically analyze the standards separately from the sample. In this embodiment of the present invention, one simply uses a previously derived standard plot to extrapolate the copy number of the current sample. This ability to re-use a standard plot reduces the number of PCR amplifications that must be carried out at any one time, permitting more samples to be evaluated within a given period of time.

In another alternative embodiment of the present invention, when a standard plot is re-used from an earlier set of standard PCR reactions, an internal standard may also be used. That is, a known standard may be spiked into the same tube as the sample. An internal standard allows minor corrections to be made for variance in the standard elution peak areas that may occur over time. The internal standard may be the same as or different than the target nucleic acid. In embodiments where the internal standard is different, it will require its own primers, which should be selected so that its PCR amplification product elution peak does not overlap with the target nucleic acid amplification products. If the internal standard is the same as target nucleic acid then its elution peak will overlap exactly with that of the target amplification products. Because of this overlap, the unknown area due to the target PCR amplification product elution peak must be calculated by first substracting out the known area of the internal standard's elution peak.

Although the method of the present invention requires each PCR reaction tube to be opened for analysis, contamination can be minimized or eliminated by using master mix additives like those used during real-time PCR. For example, the use of modified bases or analogues and specific catalytic enzymes can eliminate the possibility of cross-contamination (e.g. BRL Life Sciences Super-mix UDG). It should be noted that most MTS or HTS laboratory settings include unidirectional sample preparation and analysis, dedicated clean and dirty laboratories, separate air handling systems (HVAC), HEPA filters, and additional procedures, including staff training and designation. It also deserves mention that even in real-time PCR analysis, positive samples typically require further characterization, including sequencing and base-pair size-determination for confirmation. These also require tubes to be opened for analysis.

Although the analytical approach of the present invention requires post-PCR analysis, the method is MTS/HTS compliant, and compatible with standard automation methods. Thus, the present invention increases throughput and cost-efficiency while reducing errors, variance, or contamination. MTS or HTS compatiblity decreases the time required to analyze samples after they are obtained by reducing staff-time required to PCR screen or process multiple samples for multiple threat agents or pathogens.

The following examples illustrate that the limits of detection within the quantitative end-point PCR method are comparable to real-time PCR.

### EXAMPLES

### EXAMPLE 1

### Quantitative end-point PCR of a target nucleic acid in a sample using RP IP HPLC

Sample Preparation.

A known quantity of “target 4” was spiked onto a paper matrix and processed using standard DNA extraction procedures. The matrix and spores were disrupted by mechanical shearing and standard DNA purification was carried out using Qiagen columns and methods as specified by the vendor.

Standard Preparation.

Four standards were prepared by carrying out three serial ten-fold dilutions of an initial standard, which contained 10,000 femtograms/10 μl of the target nucleic acid (10 μl is the sample volume that was added to each reaction tube). The resulting standards contained 1000, 100 and 10 femograms/10 μl of the target nucleic acid, respectively, in the same buffer solution as the sample.

PCR Primers.

PCR primers for the target nucleic acid sequence of the simulant pathogen were designed using Primer3 software. The resulting primer sequences were oligonucleotides with 20% to 40% GC content and Tₘ=62 degrees C. Unlabeled oligonucleotide primers were synthesized using standard phosporamidite chemistry.

PCR Amplification Conditions.

PCR reaction mixtures were prepared by combining 10 μl of the target nucleic acid sample solution, or the standard solution, as prepared above, with 40 μl of a solution containing 200 nM of the forward and reverse primers and the ABI Amplitaq Gold Master Mix. The PCR reaction mixtures were placed in an ABI 7700 Prism thermal-cycler. The PCR amplification reaction included a three-step profile: a denaturing step at 95 degrees C. for 15 seconds, followed by an annealing step at 62 degrees C. for one minute, and an extension step at 68 degrees C. for 10 seconds. The reaction was repeated for 45 cycles.

Each sample or standard was subjected to a post-PCR clean-up step following the PCR reaction to remove primer-dimers and unincorporated dNTPs, primers, and polymerase. This clean-up step was carried out using Qiagen MinElute Reaction cleanup kit following the methods specified by the manufacturer.

RP IP HPLC.

The Transgenomic Inc. WAVE DNA HPLC column was used for RP IP HPLC of the PCR amplification products. All HPLC reagents and supplies used were purchased from Transgenomic Inc., including: buffers A (0.1M Tr-ethyl ammonium acetate (TEAA), pH 7.0), B (0.1M TEAA, 25% acetonitrile, pH 7.), D (75% aqueous solution of acetonitrile); the Syringe Wash solution (10% aqueous acetonitrile); and the DNASep cartridge (column).

RP IP HPLC analysis was conducted using an acetonitrile gradient in a 0.1M TEAA buffer, pH 7.0, at a constant flow rate of 0.75 mL/min. The gradient was adjusted using different ratios of buffers A and B over time (9.5 min) at constant pressure and flow-rate. The gradient was adjusted from 35% B to 61% B over 9.5 minutes to accommodate PCR amplification products varying in size from 40 to 300 base pairs using the WAVEMAKER software (ver. 4.1). The total run time including equilibration and column washing was 14 minutes. Column temperature was set to 50 degrees C. A UV detector was set to monitor elution peaks at 260 nm.

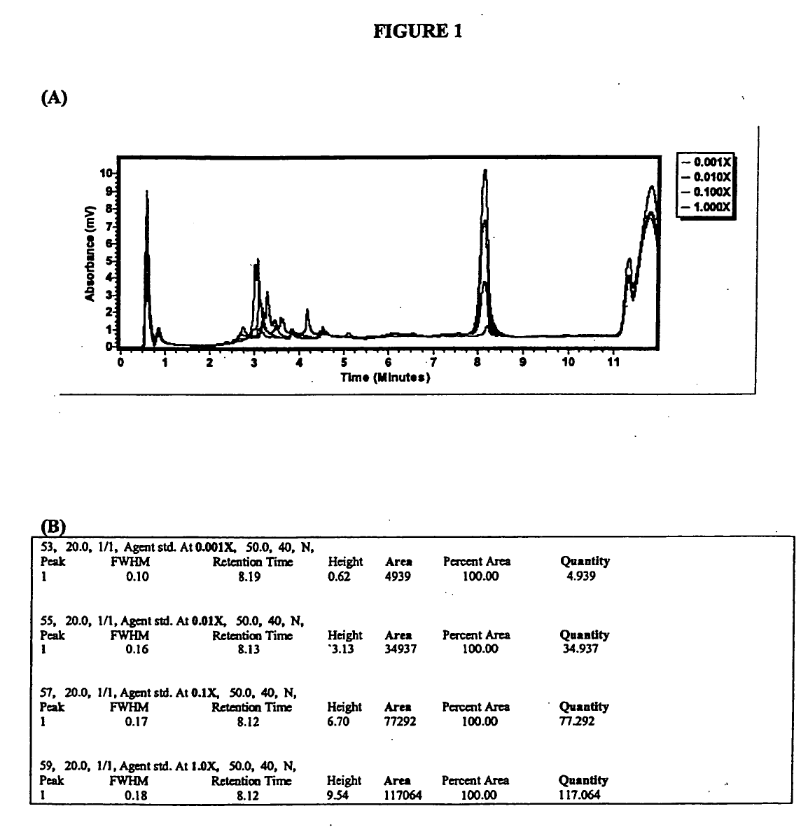

FIG. 1(A) shows an overlay of the elution profiles of the dilution series four standards of “target 4.” The elution peak at ˜8.15 minutes corresponds to the PCR amplification product of the standard. As shown in FIG. 1(B), the area of the dilution peaks for the four standard dilutions are 117065, 77292, 34937, and 4939, respectively.

Elution Peak Analysis

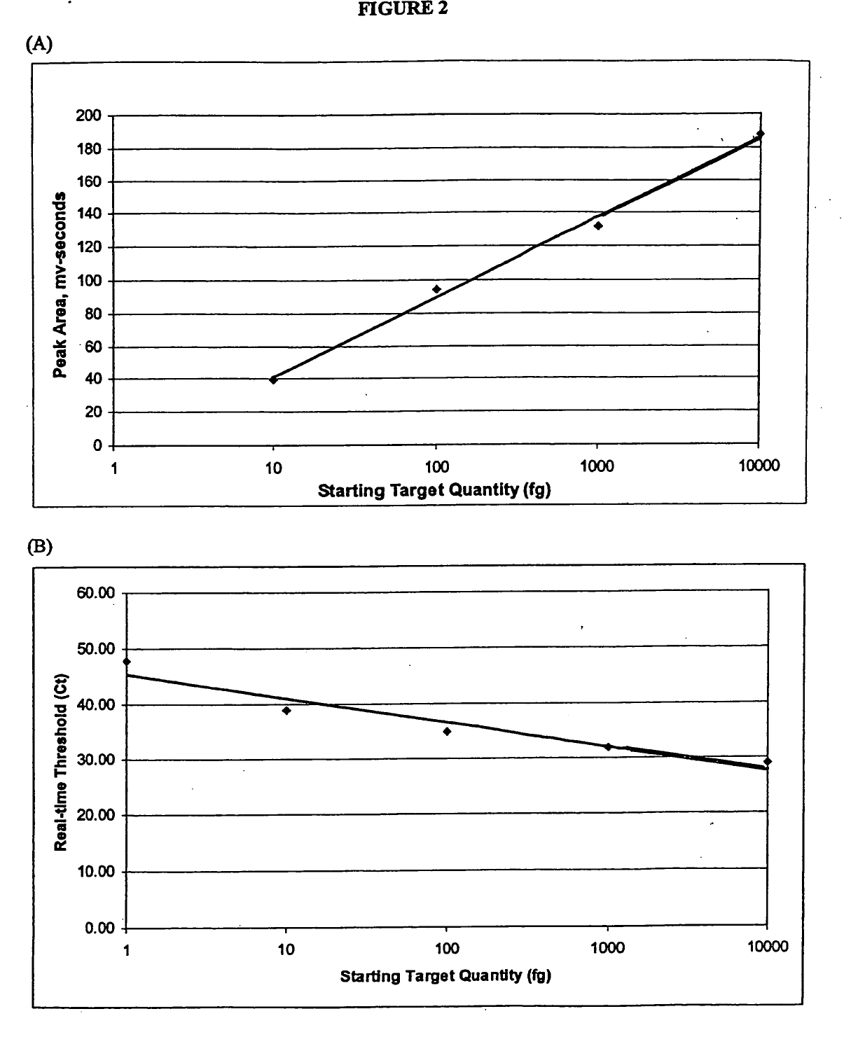

The average area of each standard elution peak was plotted versus the log (base 10) of the input amount and a plot generated by regression analysis. FIG. 2(A) shows a plot of the elution peak areas of the four standard dilutions versus the starting target nucleic acid quantity in femtograms. The plot is highly linear over the full range of standard from 10 femtograms to 10,000 femtograms. The unknown starting quantity of a “target 4” target nucleic acid sample is derived by locating its elution peak area on the plot line and extrapolating the starting target quantity from the x-axis of the plot.

Comparison to Real-Time PCR Quantitation

Real-time PCR reactions containing the same four “target 4” standards were carried out by standard methods using ABI Amplitaq Gold Master Mix. FIG. 2(B) shows a plot of the real-time threshold versus known starting target concentrations of the standards. The plot shows comparable linearity to the plot of FIG. 2(A) between 10 femtograms and 10,000 femtograms.

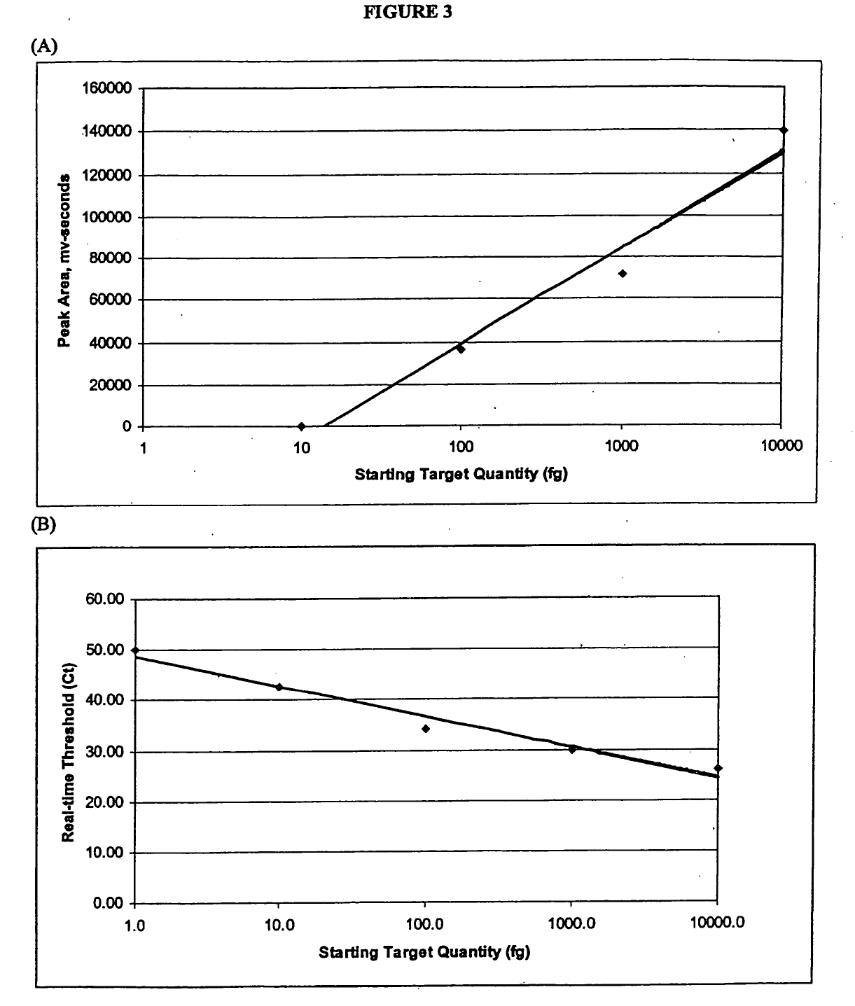

FIG. 3 shows comparable results for the quantitative analysis of a “target 3” standard dilution series carried out using the quantitative end-point PCR method (FIG. 3(A)) and the real-time PCR method (FIG. 3(B)).

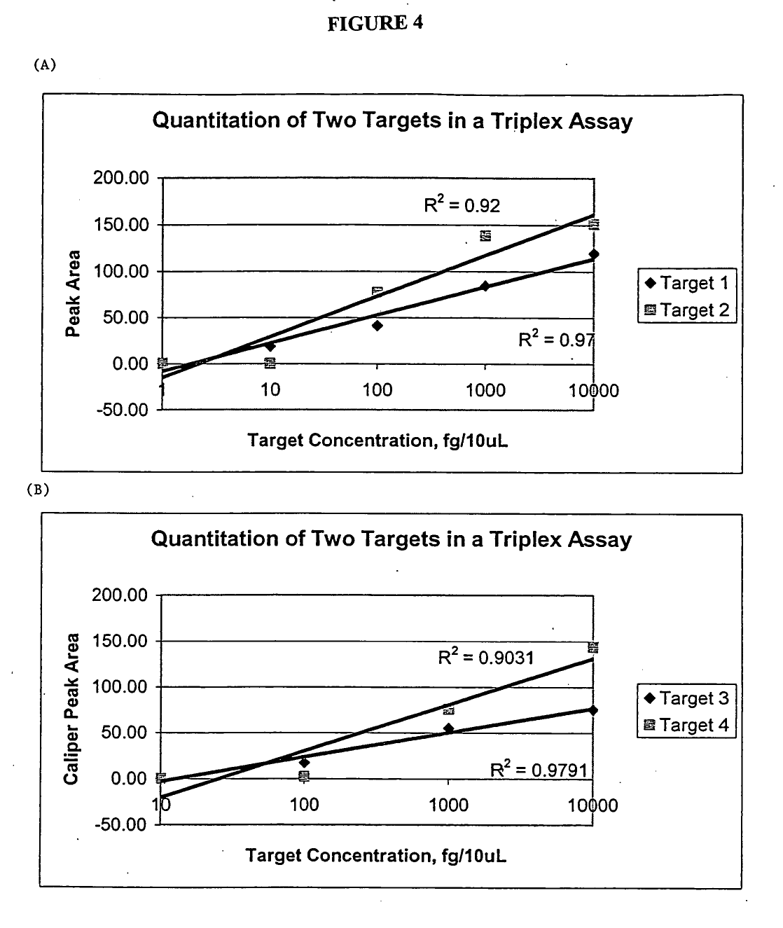

Likewise, FIG. 4 shows comparable results for the quantitative analysis of two targets in a triplex assay. The reaction mixture for this triplex assay was prepared by adding 5 μL of sanple to 20 μL of solution containing 20 nM of each primer and Invitrogen Platinum Quantitative Supermix UDG Mastermix. FIG. 4(A) shows an analysis of targets 1 and 2 using micro-channel fluidics (specifically, the Caliper AMS90SE). FIG. 4(B) shows an analysis of targets 3 and 4 using the same device.

It is understood that the examples given above are by way of illustration only and do not limit the scope of the present invention. Furthermore, numerous modifications to the present method will be readily apparent to those of skill in the art upon viewing this disclosure. It is intended that the present invention includes such modifications and is limited only by the claims that follow.

1. A method for quantifying the amount of target nucleic acids in a sample comprising:
(a) providing a sample comprising one or more target nucleic acids and one or more pairs of nucleic acid primers selected to amplify the target nucleic acids;
(b) subjecting the sample to PCR amplification conditions;
(c) introducing the sample into a separation device and detecting elution peaks of each PCR amplification product in the sample;
(d) determining the area of a PCR amplification product elution peak resulting from the amplification of one of said target nucleic acids;
(e) determining the starting amount of one of said one or more target nucleic acids in the sample by comparing the area determined in step (d) with the area of a PCR amplification product elution peak representing a PCR amplification of a standard comprising a known amount of nucleic acid; and
(f) repeating steps (d) through (e), above, for each of said one or more target nucleic acid sequences in excess of one target nucleic acid sequence.

2. The method of claim 1 wherein the amount of each target nucleic acid in the sample is determined by extrapolation from a plot comprising a first axis corresponding to a known amount of target nucleic acid in each standard and a second axis corresponding to the area of the PCR amplification product elution peak of each standard.

3. The method of claim 1 wherein the PCR amplification conditions include carrying out a PCR reaction to an end-point after the log-phase of the reaction.

4. The method of claim 1 wherein the sample comprises two to ten different target nucleic acids.

5. The method of claim 4 wherein each of the two to ten different target nucleic acids is from a different organism.

6. The method of claim 1 wherein the target nucleic acid primers are not labeled.

7. The method of claim 1 wherein the amount of the target nucleic acid in the sample is between about 10 femtograms and about 10,000 femtograms.

8. The method of claim 1 wherein the sample further comprises one or more non-target nucleic acids.

9. The method of claim 1 wherein the sample further comprises an internal standard with a nucleic acid sequence different than the target nucleic acid.

10. The method of claim 1 wherein the chromatographic separation device is a reverse phase ion-pairing HPLC column.

11. The method of claim 1 wherein the separation device is a micro-channel fluidic device.

12. The method of claim 1 wherein the length of the PCR amplification products generated from the one or more target nucleic acids is from about 75 nucleotides to about 250 nucleotides.

13. The method of claim 1 wherein the length of the PCR amplification products generated from said one or more target nucleic acids is from about 50 nucleotides to about 500 nucleotides.

14. The method of claim 1 wherein the PCR amplification products generated from two or more of said more than one target nucleic acids differ in size by at least 10 nucleotides.

15. A method for quantifying the amount of target nucleic acids in a sample comprising:
(a) providing a standard comprising a known amount of one or more target nucleic acids and one or more pairs of nucleic acid primers selected to amplify the one or more target nucleic acids;
(b) subjecting the standard to PCR amplification conditions;
(c) introducing the standard into a separation device and detecting elution peaks of each PCR amplification product resulting from the amplification of the one or more target nucleic acids;
(d) determining the area of a PCR amplification product elution peak resulting from the amplification of the one or more target nucleic acids;
(e) repeating step (d), above, for each of said one or more target nucleic acid sequences in excess of one target nucleic acid sequence included in said standard;
(f) providing a sample comprising one or more target nucleic acids and one or more pairs of nucleic acid primers selected to amplify the target nucleic acids;
(g) subjecting the sample to PCR amplification conditions;
(h) introducing the sample into a chromatographic separation device and detecting elution peaks of each PCR amplification product in the sample;
(i) determining the area of a PCR amplification product elution peak resulting from the amplification of one of said target nucleic acids;
(j) determining the starting amount of one of said one or more target nucleic acids in the sample by comparing the area determined in step (i) with the area determined in step (d); and
(k) repeating steps (i) through (j), above, for each of said one or more target nucleic acid sequences in excess of one target nucleic acid sequence.

16. The method of claim 15 wherein the amount of each target nucleic acid in the sample is determined by extrapolation from a plot comprising a first axis corresponding to a known amount of target nucleic acid in each standard and a second axis corresponding to the area of the PCR amplification product elution peak of each standard.

17. The method of claim 15 wherein the sample further comprises one or more non-target nucleic acids.

18. The method of claim 15 wherein the sample further comprises an internal standard with a nucleic acid sequence different than the target nucleic acid.

19. The method of claim 15 wherein the separation device is a reverse phase ion-pairing HPLC column.

20. The method of claim 15 wherein the separation device is a micro-channel fluidic device.

21. A method for quantifying the amount of target nucleic acid in a sample comprising determining the area under the curve of an elution peak provided by separation analysis of a PCR amplification product resulting from said target nucleic acid and extrapolating the starting amount of the target nucleic acid in said sample by comparing said area under the curve with a standard.

22. The method of claim 21 wherein the amount of each target nucleic acid in the sample is determined by extrapolation from a plot comprising a first axis corresponding to a known amount of target nucleic acid in each standard and a second axis corresponding to the area of the PCR amplification product elution peak of each standard.

23. The method of claim 21 wherein the sample further comprises one or more non-target nucleic acids.

24. The method of claim 21 wherein the sample further comprises an internal standard with a nucleic acid sequence different than the target nucleic acid.

25. The method of claim 21 wherein the separation analysis is carried out using a reverse phase ion-pairing HPLC column.

26. The method of claim 21 wherein the separation analysis is carried out using a micro-channel fluidic device.

27. A method for quantifying the amount of target nucleic acids in a sample comprising:
(a) providing a sample comprising four target nucleic acids and four pairs of nucleic acid primers selected to amplify the target nucleic acids;
(b) subjecting the sample to PCR amplification conditions;
(c) introducing the sample into a reverse phase ion-pairing HPLC column and detecting elution peaks of each PCR amplification product in the sample;
(d) determining the area of a PCR amplification product elution peak resulting from the amplification of one of said four target nucleic acids;
(e) determining the starting amount of one of said four target nucleic acids in the sample by comparing the area determined in step (d) with the area of a PCR amplification product elution peak representing a PCR amplification of a standard comprising a known amount of nucleic acid; and
(f) repeating steps (d) through (e), above, for each of said four target nucleic acids.

28. A method for quantifying the amount of target nucleic acids in a sample comprising:
(a) providing a sample comprising six target nucleic acids and six pairs of nucleic acid primers selected to amplify the target nucleic acids;
(b) subjecting the sample to PCR amplification conditions;
(c) introducing the sample into a reverse phase ion-pairing HPLC column and detecting elution peaks of each PCR amplification product in the sample;
(d) determining the area of a PCR amplification product elution peak resulting from the amplification of one of said six target nucleic acids;
(e) determining the starting amount of one of said six target nucleic acids in the sample by comparing the area determined in step (d) with the area of a PCR amplification product elution peak representing a PCR amplification of a standard comprising a known amount of nucleic acid; and
(f) repeating steps (d) through (e), above, for each of said six target nucleic acids.
